(19) United States
(12) Patent Application Publication
(10) Pub. No.: US 2005/0181043 A1
(43) Pub. Date: Aug. 18, 2005

(54) Alendronate salt tablet compositions

(76) Inventors: Indranil Nandi, Plainsboro, NJ (US); Pablo Davila, East Windsor, NJ (US); Theodore Edward Burnell, Superior, CO (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/779,050

(22) Filed: Feb. 12, 2004

(51) Int. Cl. A61K 31/66; A61K 9/20

(52) U.S. Cl. 424/464; 514/102

(57) ABSTRACT

A tablet composition comprising an alendronate salt and optionally one or more excipients, wherein the cumulative total of the alendronate salt particles in the composition have a particle size distribution as follows: (a) about 19% to about 25% of the particles have a particle size of 250 microns as determined by a U.S. Sieve No. 60; (b) about 14% to about 17% of the particles have a particle size of 180 microns as determined by a U.S. Sieve No. 80; (c) about 10% to about 13% of the particles have a particle size of about 150 microns as determined by a U.S. Sieve No. 100; (d) about 16% to about 23% of the particles have a particle size of 90 microns as determined by a U.S. Sieve No. 170; (e) about 9% to about 17% of the particles have a particle size of 75 microns as determined by a U.S. Sieve No. 250; (f) about 13% to about 16% of the particles have a particle size of 45 microns as determined by a U.S. Sieve No. 325; and (g) about 1% to about 10% of the particles have a particle size of less than 45 microns, wherein the weight percents are based on the total weight of the alendronate salt. The tablets do not require lactose or polyvinylpyrrolidone in order to achieve blend uniformity.

### FIELD OF THE INVENTION

The present invention provides a tablet composition comprising an alendronate salt.

### BACKGROUND OF THE INVENTION

Alendronate sodium acts as a specific inhibitor of osteoclast-mediated bone resorption and it is a synthetic analog of pyrophosphate that binds to the hydroxyapatite found in bone. Alendronate sodium is chemically described as (4-amino-1-hydroxybutylidene)bisphosphonic acid monosodium salt trihydrate. Alendronate sodium is commercially-available under the trademark FOSAMAX® from Merck. FOSAMAX® is supplied as tablets containing 6.53, 13.05, 45.68, 52.21 and 91.37 mg of alendronate monosodium salt trihydrate, which is the molar equivalent of 5,10, 35, 40 and 70 mg, respectively, of free acid, and the following inactive ingredients: microcrystalline cellulose, anhydrous lactose, croscarmellose sodium and magnesium stearate. Tablets of 10 mg FOSAMAX® also contain carnuba wax.

Several prior art documents describe pharmaceutical compositions containing alendronate sodium and processes for the preparation thereof. Such documents include U.S. Pat. Nos. 5,358,941; 5,681,590; 5,849,726; 6,008,207; 6,090,410; and 6,194,004.

### SUMMARY OF THE INVENTION

The invention provides a tablet composition comprising an alendronate salt and optionally one or more excipients, wherein the cumulative total of the alendronate salt particles in the composition have a particle size distribution as follows:
(a) about 19% to about 25% of the particles have a particle size of 250 microns as determined by a U.S. Sieve No. 60;
(b) about 14% to about 17% of the particles have a particle size of 180 microns as determined by a U.S. Sieve No. 80;
(c) about 10% to about 13% of the particles have a particle size of about 150 microns as determined by a U.S. Sieve No. 100;
(d) about 16% to about 23% of the particles have a particle size of 90 microns as determined by a U.S. Sieve No.170;
(e) about 9% to about 17% of the particles have a particle size of 75 microns as determined by a U.S. Sieve No. 250;
(f) about 13% to about 16% of the particles have a particle size of 45 microns as determined by a U.S. Sieve No. 325; and
(g) about 1 % to about 10% of the particles have a particle size of less than 45 microns, wherein the weight percents are based on the total weight of the alendronate salt.

According to another aspect, the invention provides a tablet composition, as described above, that is essentially free of lactose.

According to another aspect, the invention provides a tablet composition, as described above, that is essentially free of polyvinylpyrrolidone.

According to another aspect, the invention provides a dry blending method for preparing a tablet composition comprising an alendronate salt and optionally one or more excipients, said method comprising: (i) mixing an alendronate salt and optionally one or more excipients; and (ii) compressing the mixture to form a tablet, wherein the cumulative total of the alendronate salt particles in the composition have a particle size distribution as follows:
(a) about 19% to about 25% of the particles have a particle size of 250 microns as determined by a U.S. Sieve No.60;
(b) about 14% to about 17% of the particles have a particle size of 180 microns as determined by a U.S. Sieve No. 80;
(c) about 10% to about 13% of the particles have a particle size of about 150 microns as determined by a U.S. Sieve No.100;
(d) about 16% to about 23% of the particles have a particle size of 90 microns as determined by a U.S. Sieve No.170;
(e) about 9% to about 17% of the particles have a particle size of 75 microns as determined by a U.S. Sieve No. 250;
(f) about 13% to about 16% of the particles have a particle size of 45 microns as determined by a U.S. Sieve No. 325; and
(g) about 1 % to about 10% of the particles have a particle size of less than 45 microns,
   wherein the weight percents are based on the total weight of the alendronate salt.

The present inventors have unexpectedly determined that tablets containing an alendronate salt having a specified particle size distribution, wherein the tablets are prepared by a dry blending method, do not require lactose or polyvinylpyrrolidone in order to achieve blend uniformity.

### DETAILED DESCRIPTION OF THE INVENTION

The invention provides a tablet composition comprising an alendronate salt and optionally one or more excipients, wherein the cumulative total of the alendronate salt particles in the composition have a particle size distribution as follows:
(a) about 19% to about 25%, preferably about 20% to about 23%, of the particles have a particle size of 250 microns as determined by a U.S. Sieve No. 60;
(b) about 14% to about 17%, preferably about 15% to about 16%, of the particles have a particle size of 180 microns as determined by a U.S. Sieve No. 80;
(c) about 10% to about 13%, preferably about 11 % to about 12%, of the particles have a particle size of about 150 microns as determined by a U.S. Sieve No.100;
(d) about 16% to about 23%, preferably about 17% to about 21%, of the particles have a particle size of 90 microns as determined by a U.S. Sieve No.170;
(e) about 9% to about 17%, preferably about 10% to about 16%, of the particles have a particle size of 75 microns as determined by a U.S. Sieve No. 250;
(f) about 13% to about 16%, preferably about 14% to about 15%, of the particles have a particle size of 45 microns as determined by a U.S. Sieve No. 325; and
(g) about 1 % to about 10%, preferably about 3% to about 8%, of the particles have a particle size of less than 45 microns,
   wherein the weight percents are based on the total weight of the alendronate salt.

In one embodiment of the invention, the cumulative total of the alendronate salt particles in the composition have a particle size distribution as follows:
(a) about 20.1 % of the particles have a particle size of 250 microns as determined by a U.S. Sieve No.60;
(b) about 15% of the particles have a particle size of 180 microns as determined by a U.S. Sieve No. 80;
(c) about 11.2% of the particles have a particle size of about 150 microns as determined by a U.S. Sieve No.100;
(d) about 20.5% of the particles have a particle size of 90 microns as determined by a U.S. Sieve No.170;
(e) about 15.2% of the particles have a particle size of 75 microns as determined by a U.S. Sieve No. 250;
(f) about 14.6% of the particles have a particle size of 45 microns as determined by a U.S. Sieve No. 325; and
(g) about 3.1% of the particles have a particle size of less than 45 microns.

In one embodiment of the invention, the cumulative total of the alendronate salt particles in the composition have a particle size distribution as follows:
(a) about 22.9% of the particles have a particle size of 250 microns as determined by a U.S. Sieve No.60;
(b) about 15.6% of the particles have a particle size of 180 microns as determined by a U.S. Sieve No. 80;
(c) about 11.5% of the particles have a particle size of about 150 microns as determined by a U.S. Sieve No.100;
(d) about 17.5% of the particles have a particle size of 90 microns as determined by a U.S. Sieve No.170;
(e) about 10.6% of the particles have a particle size of 75 microns as determined by a U.S. Sieve No. 250;
(f) about 14.8% of the particles have a particle size of 45 microns as determined by a U.S. Sieve No. 325; and
(g) about 7.3% of the particles have a particle size of less than 45 microns.

As used herein, “alendronate salt” refers to a salt of (4-amino-1-hydroxybutylidene)bisphosphonic acid. Preferably, the alendronate salt is (4-amino-1-hydroxybutylidene)bisphosphonic acid monosodium salt trihydrate.

The alendronate salt is present in the tablet compositions in an amount of from about 1 weight percent (wt. %) to about 100 wt. %, based on the total weight of the composition. Preferably, the alendronate salt is present in an amount of from about 30 wt. % to about 50 wt. %, more preferably about 40 wt. %, based on the total weight of the composition.

The tablet compositions may contain one or more excipients. Examples of excipients include, but are not limited to, diluents, disintegrants, lubricants, glidants, binders, fillers, emulsifiers, electrolytes, wetting agents, solubilizers, surfactants, colors, pigments and anti-caking agents. A combination of excipients may also be used. Preferably, the excipients meet the standards of the National Formulary (“NF”) or United States Pharmacopoeia (“USP”).

Examples of glidants include, but are not limited to, silica, magnesium trisilicate, powdered cellulose, starch, talc and tribasic calcium phosphate. A preferred glidant is silicon dioxide.

Examples of fillers or diluents include, but are not limited to, spray-dried or anhydrous lactose; sucrose; dextrose; starch; pre-gelatinized starch; polyols, such as mannitol, sorbitol and xylitol; cellulose, such as microcrystalline cellulose; and inorganic salts, such as dibasic calcium phosphate, tribasic calcium phosphate and calcium sulfate. Preferably, the filler or diluent is microcrystalline cellulose.

Examples of disintegrants include, but are not limited to, starch and starch derivatives, including cross-linked sodium salt of a carboxymethyl ether of starch, such as sodium starch glycolate (Primojel®); pre-gelatinized starch, such as Starch 1500; sodium starch glycolate; cross-linked sodium carboxymethyl cellulose, such as croscarmellose sodium; cross-linked polyvinylpyrrolidone, such as crospovidone; and microcrystalline cellulose. A preferred disintegrant is sodium starch glycolate.

Examples of binders include, but are not limited to, cellulose derivatives, such as microcrystalline cellulose, methylcellulose, carboxymethycellulose sodium, hydroxypropyl methylcellulose, hydroxyethyl cellulose and hydroxypropyl cellulose; polyvidone; polyvinyl pyrrolidone; gelatin; natural gums, such as acacia, tragacanth, guar and pectin; starch paste; pre-gelatinized starch; sucrose; corn syrup; polyethylene glycols and sodium alginate; ammonium calcium alginate; magnesium aluminum silicate; and polyethylene glycols. A preferred binder is pre-gelatinized starch.

Several co-processed filler-binders are commercially-available, including cellactose (α-lactose monohydrate and powdered cellulose 75:25), microcelac (α-lactose monohydrate and powdered cellulose 75:25), ludipress (93% ═-lactose monohydrate, 3.5% polyvinylpyrrolidone and 3.5% crospovidone) and pharmatose DCL 40 (95% β-lactose and 5% lactitol).

Examples of lubricants include, but are not limited to, vegetable oils, such as hydrogenated vegetable oil or hydrogenated castor oil; polyethylene glycols, such as PEG-4000 and PEG-6000; stearic acid; salts of stearic acid, such as magnesium stearate, sodium stearate and sodium stearyl fumarate; mineral salts, such as talc; inorganic salts; organic salts, such as sodium benzoate, sodium acetate and sodium oleate; and polyvinyl alcohols. A preferred lubricant is magnesium stearate.

In a preferred embodiment of the invention, the tablet composition is prepared with the following excipients: sodium starch glycolate as the disintegrant, microcrystalline cellulose as the filler, and magnesium stearate as the lubricant.

It is within the scope of the invention to prepare the tablet compositions of the invention that are “essentially free of lactose”. As used herein, “essentially free of lactose” means that the compositions contain less than 3 wt. %, preferably less than 1 wt. %, of lactose, based on the total weight of the composition.

It is within the scope of the invention to prepare the tablet compositions of the invention that are “essentially free of polyvinylpyrrolidone”. As used herein, “essentially free of polyvinylpyrrolidone” means that the compositions contain less than 3 wt. %, preferably less than 1 wt. %, of polyvinylpyrrolidone, based on the total weight of the composition.

In one embodiment of the invention, the tablet compositions are prepared by a dry blending method. The dry blending method comprises: (i) mixing an alendronate salt and optionally one or more excipients; and (ii) compressing the mixture in a Manestry beta press to form a tablet. Blenders appropriate for large scale dry blending include twin shell blenders, double cone blenders, V-blenders or bin-blenders. A preferred blender is a V-shell PK blender. High-speed, high-shear mixers may also be used. The tablets of the invention may optionally be coated with a functional coating, such as an enteric coating, or a non-functional coating for appearance.

The tablet compositions of the invention are especially useful in the treatment of osteoporosis, prevention of bone loss, and malignant hypercalcemia.

The following non-limiting examples illustrate further aspects of the invention.

### EXAMPLE 1

Preparation of Alendronate Salt Tablets Containing 5 mg of Alendronate.

| | Amount per Tablet | |
|---|---|---|
| Ingredient | % | Mg per Tablet |
| Alendronate Monosodium Salt Trihydrate | 5.2% | 6.53 mg |
| Microcrystalline Cellulose NF (Avicel PH 102) | 91.3% | 114.1 mg |
| Sodium Starch Glycolate NF (Primojel) | 3.0% | 3.75 mg |
| Magnesium Stearate NF | 0.5% | 0.63 mg |
| Total Weight | 100% | 125 mg |

The alendronate salt had the following particle size distribution: (a) about 20.1% of the particles have a particle size of 250 microns as determined by a U.S. Sieve No.60; (b) about 15% of the particles have a particle size of 180 microns as determined by a U.S. Sieve No.80; (c) about 11.2% of the particles have a particle size of about 150 microns as determined by a U.S. Sieve No.100; (d) about 20.5% of the particles have a particle size of 90 microns as determined by a U.S. Sieve No.170; (e) about 15.2% of the particles have a particle size of 75 microns as determined by a U.S. Sieve No. 250; (f) about 14.6% of the particles have a particle size of 45 microns as determined by a U.S. Sieve No. 325; and (g) about 3.1 % of the particles have a particle size of less than 45 microns, wherein the weight percents are based on the total weight of the alendronate salt.

The alendronate salt, microcrystalline cellulose, and sodium starch glycolate were mixed using a Patterson Kelly V-blender to form a uniform dry mixture. Magnesium stearate was added to the mixture and the resulting mixture was mixed using the Patterson Kelly V-blender. The mixture was compressed into tablets.

### EXAMPLE 2

Preparation of Alendronate Salt Tablets Containing 10 mg of Alendronate.

| | Amount per Tablet | |
|---|---|---|
| Ingredient | % | Mg per Tablet |
| Alendronate Monosodium Salt Trihydrate | 5.2% | 13.06 mg |
| Microcrystalline Cellulose NF (Avicel PH 102) | 91.3% | 228.19 mg |
| Sodium Starch Glycolate NF (Primojel) | 3.0% | 7.5 mg |
| Magnesium Stearate NF | 0.5% | 1.25 mg |
| Total Weight | 100% | 250 mg |

The tablets were prepared according to the procedure set forth in Example 1.

The ingredients prior to compression in Example 2 were evaluated in a blend uniformity study. After mixing all of the ingredients in the Patterson Kelly V-Blender, ten samples of the mixture were removed from different locations in the blender and a potency assay was determined by HPLC. The results of the potency assay was as follows: average of ten samples was 106.5, range 103.5 to 108.2, and a relative standard deviation (RSD) of 1.5. Thus, the mixture displayed consistent blend uniformity.

### EXAMPLE 3

Preparation of Alendronate Salt Tablets Containing 35 mg of Alendronate.

| | Amount per Tablet | |
|---|---|---|
| Ingredient | % | Mg per Tablet |
| Alendronate Monosodium Salt Trihydrate | 26.1% | 45.68 mg |
| Microcrystalline Cellulose NF (Avicel PH 102) | 70.4% | 123.2 mg |
| Sodium Starch Glycolate NF (Primojel) | 3.0% | 5.25 mg |
| Magnesium Stearate NF | 0.5% | 0.87 mg |
| Total Weight | 100% | 175 mg |

The tablets were prepared according to the procedure set forth in Example 1.

### EXAMPLE 4

Preparation of Alendronate Salt Tablets Containing 40 mg of Alendronate.

| | Amount per Tablet | |
|---|---|---|
| Ingredient | % | Mg per Tablet |
| Alendronate Monosodium Salt Trihydrate | 26.1% | 52.21 mg |
| Microcrystalline Cellulose NF (Avicel PH 102) | 70.4% | 140.79 mg |
| Sodium Starch Glycolate NF (Primojel) | 3.0% | 6.0 mg |
| Magnesium Stearate NF | 0.5% | 1.0 mg |
| Total Weight | 100% | 200 mg |

The tablets were prepared according to the procedure set forth in Example 1.

### EXAMPLE 5

Preparation of Alendronate Salt Tablets Containing 70 mg of Alendronate.

| | Amount per Tablet | |
|---|---|---|
| Ingredient | % | Mg per Tablet |
| Alendronate Monosodium Salt Trihydrate | 26.1% | 91.37 mg |
| Microcrystalline Cellulose NF (Avicel PH 102) | 70.4% | 246.38 mg |
| Sodium Starch Glycolate NF (Primojel) | 3.0% | 10.5 mg |
| Magnesium Stearate NF | 0.5% | 1.75 mg |
| Total Weight | 100% | 350 mg |

The tablets were prepared according to the procedure set forth in Example 1.

While the invention has been described with particular reference to certain embodiments thereof, it will be understood that changes and modifications may be made by those of ordinary skill within the scope and spirit of the following claims:

1. A tablet composition comprising an alendronate salt and optionally one or more excipients, wherein the cumulative total of the alendronate salt particles in the composition have a particle size distribution as follows:
(a) about 19% to about 25% of the particles have a particle size of 250 microns as determined by a U.S. Sieve No. 60;
(b) about 14% to about 17% of the particles have a particle size of 180 microns as determined by a U.S. Sieve No. 80;
(c) about 10% to about 13% of the particles have a particle size of about 150 microns as determined by a U.S. Sieve No.100;
(d) about 16% to about 23% of the particles have a particle size of 90 microns as determined by a U.S. Sieve No.170;
(e) about 9% to about 17% of the particles have a particle size of 75 microns as determined by a U.S. Sieve No. 250;
(f) about 13% to about 16% of the particles have a particle size of 45 microns as determined by a U.S. Sieve No. 325; and
(g) about 1% to about 10% of the particles have a particle size of less than 45 microns, wherein the weight percents are based on the total weight of the alendronate salt.

2. A tablet composition comprising an alendronate salt and optionally one or more excipients, wherein the cumulative total of the alendronate salt particles in the composition have a particle size distribution as follows:
(a) about 20% to about 23% of the particles have a particle size of 250 microns as determined by a U.S. Sieve No. 60;
(b) about 15% to about 16% of the particles have a particle size of 180 microns as determined by a U.S. Sieve No. 80;
(c) about 11 % to about 12% of the particles have a particle size of about 150 microns as determined by a U.S. Sieve No.100;
(d) about 17% to about 21% of the particles have a particle size of 90 microns as determined by a U.S. Sieve No.170;
(e) about 10% to about 16% of the particles have a particle size of 75 microns as determined by a U.S. Sieve No. 250;
(f) about 14% to about 15% of the particles have a particle size of 45 microns as determined by a U.S. Sieve No. 325; and
(g) about 3% to about 8% of the particles have a particle size of less than 45 microns, wherein the weight percents are based on the total weight of the alendronate salt.

3. A tablet composition comprising an alendronate salt and optionally one or more excipients, wherein the cumulative total of the alendronate salt particles in the composition have a particle size distribution as follows:
(a) about 20.1% of the particles have a particle size of 250 microns as determined by a U.S. Sieve No. 60;
(b) about 15% of the particles have a particle size of 180 microns as determined by a U.S. Sieve No. 80;
(c) about 11.2% of the particles have a particle size of about 150 microns as determined by a U.S. Sieve No.100;
(d) about 20.5% of the particles have a particle size of 90 microns as determined by a U.S. Sieve No.170;
(e) about 15.2% of the particles have a particle size of 75 microns as determined by a U.S. Sieve No. 250;
(f) about 14.6% of the particles have a particle size of 45 microns as determined by a U.S. Sieve No. 325; and
(g) about 3.1% of the particles have a particle size of less than 45 microns, wherein the weight percents are based on the total weight of the alendronate salt.

4. A tablet composition comprising an alendronate salt and optionally one or more excipients, wherein the cumulative total of the alendronate salt particles in the composition have a particle size distribution as follows:
(a) about 22.9% of the particles have a particle size of 250 microns as determined by a U.S. Sieve No.60;
(b) about 15.6% of the particles have a particle size of 180 microns as determined by a U.S. Sieve No.80;
(c) about 11.5% of the particles have a particle size of about 150 microns as determined by a U.S. Sieve No.100;
(d) about 17.5% of the particles have a particle size of 90 microns as determined by a U.S. Sieve No.170;
(e) about 10.6% of the particles have a particle size of 75 microns as determined by a U.S. Sieve No. 250;
(f) about 14.8% of the particles have a particle size of 45 microns as determined by a U.S. Sieve No. 325; and
(g) about 7.3% of the particles have a particle size of less than 45 microns, wherein the weight percents are based on the total weight of the alendronate salt.

5. The composition according to claim 1, wherein the alendronate salt is (4-amino-1-hydroxybutylidene)bisphosphonic acid monosodium salt trihydrate.

6. The composition according to claim 1, wherein the alendronate salt is present in an amount of from about 1 weight percent to about 100 weight percent, based on the total weight of the composition.

7. The composition according to claim 6, wherein the alendronate salt is present in an amount of from about 30 weight percent to about 50 weight percent, based on the total weight of the composition.

8. The composition according to claim 1, wherein the excipient is selected from the group consisting of diluents, disintegrants, lubricants, glidants, binders, fillers, emulsifiers, electrolytes, wetting agents, solubilizers, surfactants, colors, pigments, anti-caking agents and combinations thereof.

9. The composition according to claim 1, wherein the excipients are sodium starch glycolate, microcrystalline cellulose and magnesium stearate.

10. The composition according to claim 1, which is essentially free of lactose.

11. The composition according to claim 1, which is essentially free of polyvinylpyrrolidone.

12. A dry blending method for preparing a tablet composition comprising an alendronate salt and optionally one or more excipients, said method comprising: (i) mixing an alendronate salt and optionally one or more excipients; and (ii) compressing the mixture to form a tablet, wherein the cumulative total of the alendronate salt particles in the composition have a particle size distribution as follows:
(a) about 19% to about 25% of the particles have a particle size of 250 microns as determined by a U.S. Sieve No. 60;
(b) about 14% to about 17% of the particles have a particle size of 180 microns as determined by a U.S. Sieve No. 80;
(c) about 10% to about 13% of the particles have a particle size of about 150 microns as determined by a U.S. Sieve No.100;
(d) about 16% to about 23% of the particles have a particle size of 90 microns as determined by a U.S. Sieve No.170;
(e) about 9% to about 17% of the particles have a particle size of 75 microns as determined by a U.S. Sieve No. 250;
(f) about 13% to about 16% of the particles have a particle size of 45 microns as determined by a U.S. Sieve No. 325; and
(g) about 1% to about 10% of the particles have a particle size of less than 45 microns, wherein the weight percents are based on the total weight of the alendronate salt.

13. A method of treating a disease or disorder in a subject in need thereof comprising administering to the subject a therapeutically effective amount of the tablet composition according to claim 1.

14. A pharmaceutical package comprising a high-density polyethylene bottle which comprises the tablet composition according to claim 1.
